(19) United States
(12) Patent Application Publication
(10) Pub. No.: US 2025/0369891 A1
(43) Pub. Date: Dec. 4, 2025

(54) METHOD AND DEVICE FOR PREPARING A SERS SUBSTRATE FOR ANALYSIS OF AN ANALYTE IN A FLUID SAMPLE AND USE OF A NANOPARTICLE COATED CATCH FILTER AS SERS SUBSTRATE

(71) Applicant: NOSTICS B.V., Amsterdam (NL)

(72) Inventors: Rochelle Alexia NIEMEIJER, Delft (NL); Duco Martijn BLANKEN, Delft (NL); Clemens Johannes VAN DE SLUIS, Delft (NL)

(73) Assignee: NOSTICS B.V., Amsterdam (NL)

(21) Appl. No.: 18/874,443

(22) PCT Filed: Jun. 14, 2023
(86) PCT No.: PCT/NL2023/050331

§ 371 Date: Dec. 12, 2024

(30) Foreign Application Priority Data
Jun. 14, 2022 (NL) 2032167

(51) Int. Cl. G01N 21/65; G01N 1/40; G01N 33/50

(52) U.S. Cl. CPC G01N 21/658; G01N 1/4077; G01N 33/5005; G01N 2001/4088

(57) ABSTRACT

The invention relates to a method for preparing a SERS substrate for analyte analysis in a fluid sample. The method comprises the steps of providing at least one fluid sample, filtering said sample and converting said filter into a SERS substrate by applying a coating step.

The invention relates to a method for preparing a SERS substrate for analysis of an analyte in a fluid sample, for example pathogen analysis in a body fluid sample. The invention also relates to the use of at least one nanoparticle coated catch filter as SERS substrate in a Raman spectroscopy step. The invention further relates to a device for preparing a SERS substrate for analysis of an analyte in a fluid sample.

Medicaments, such as antibiotics, are rarely prescribed to humans and/or animals showing symptoms of illness based upon a definitive diagnosis since there is often not sufficient time to await test results or because expensive laboratory testing equipment is not available. In particular when it comes to the prescription of antibiotics, diagnostic testing is of major importance. Diagnostic testing can show whether or not an antibiotic is actually needed and more in particular which type of antibiotic is to be applied. Standard laboratory testing of body fluid samples takes typically 2 to 4 days due to the time required to collect the sample, send it to the lab and the processing thereof. One of the most time consuming steps in this analysis process is the cultivation of pathogens in the body fluid sample which typically takes at least 10 hours. The cultivation step is often needed to be able to collect sufficient concentration of the pathogens for further analysis by the standard diagnostics tests, such as MALDI-TOF, PCR or novel techniques, such as deposition on a Surface-Enhanced Raman Spectroscopy (SERS) substrate for analysis in a Raman spectroscopy step. If no cultivation is applied, the diagnostic step takes typically considerably longer. During the Raman spectroscopy step, the presence of any pathogens can be analysed and if present, the pathogens could be identified. As said, since there is often no possibility to await test results, antibiotics will be prescribed as a precaution prior to a definitive diagnosis is set. However, a consequence of the over prescription of antibiotics is antimicrobial resistance, which is the development of resistance to existing antibiotics which in turn makes us unable to treat infections. This is highly undesirable as it may cause a critical risk for public health. Hence, there is a serious need to be able to quickly and cheaply detect and identify the right pathogen in infections in order to offer the correct treatment to patients (human and/or animal) as well as preserving antibiotics for the infections that need them the most.

It is a goal of the invention to at least partially overcome the abovementioned problem and in particular to contribute to the provision of rapid and low-cost diagnostic and analysis solutions.

The invention provides thereto a method for preparing a SERS substrate for analysis of at least one analyte in a fluid sample, comprising the steps of:
providing at least one fluid sample;
optionally, filtering the fluid sample through at least one coarse filter, wherein the coarse filter is configured to remove at least a fraction of particles from the fluid sample, in particular particles which are larger than at least one analyte;
filtering the fluid sample through at least one catch filter, wherein the catch filter is configured to capture at least a fraction of at least one analyte if present in the fluid sample; and
filtering at least one solution comprising nanoparticles through the catch filter such that at least part of the catch filter will be or is coated with nanoparticles thereby forming a SERS substrate.

A preferred application of the method and device according to the present invention is related to pathogen analysis in a body fluid sample. The invention provides thereto in a preferred embodiment a method for preparing a SERS substrate for pathogen analysis in a sample, in particular a body fluid sample, comprising the steps of:
providing at least one sample, in particular a body fluid sample;
optionally filtering or flushing the sample, in particular the body fluid sample through at least one coarse filter, wherein the coarse filter is configured to remove at least a fraction of particles from the sample, in particular body fluid sample, in particular particles which are larger than pathogens;
filtering or flushing the sample, in particular the body fluid sample, through at least one catch filter, wherein the catch filter is configured to capture pathogens if present in the sample, in particular in the body fluid sample;
filtering or flushing at least one solution comprising nanoparticles through the catch filter such that at least part of the catch filter will be covered and/or coated with nanoparticles thereby forming a SERS substrate.

The method according to the present invention provides a creative and efficient way wherein samples, in particular fluid samples, such as body fluid samples, can be analysed upon the presence of analytes, such as pathogens and the identification thereof. The sample being flushed through at least one catch filter which is configured to capture analytes, such as pathogens, if present in the sample enables that at least a fraction of the analytes, in particular pathogens, and preferably substantially all pathogens, present in the (body) fluid sample will be captured. Subsequently, the step wherein at least one solution comprising nanoparticles is filtered through the catch filter enables that at least part of the catch filter will be (directly or indirectly) coated with nanoparticles. In fact, this step achieves that a 3D mesh of the catch filter, any captured pathogen and nanoparticles is obtained. In this way, the at least partially coated catch filter forms a SERS substrate which can be applied in Raman spectroscopy. Due to the wet coating step, wherein the catch filter is flushed with a solution comprising nanoparticles, a randomized pattern of analytes, for example pathogen-if present-and nanoparticles is obtained. It was experimentally found that such 3D randomized and 3D configuration of nanoparticles upon the catch filter applied for target particle filtration, or pathogen filtration, is useable as SERS substrate in Raman spectroscopy. In the method according to the present invention, the analytes, such as pathogens (if present in the sample) are collected prior to the SERS substrate is formed and are thus embedded within the SERS substrate. This results in enhanced possibilities to analyse the presence of any analytes, such as pathogens. Capturing the analytes, such as pathogens in a (catch) filter allows to concentrate dilute analytes, such as pathogens on a localized surface. Therefore, there is no need to cultivate the captured analytes, such as pathogens to increase their concentration when applying the method according to the present invention. Typically, in case the analytes are pathogens, the cultivation of pathogens takes at least 10 hours. This time can be crucial when it is to decide if and how to treat a patient with medicaments such as antibiotics. The method according to the invention thus benefits of a significantly reduced amount of time which is needed before samples, such as body fluid samples, can be analysed. It is non-obvious to coat the catch filter with nanoparticles after the (body) fluid sample is filtered and apply (at least part of) the filter subsequently as basis in the Raman spectroscopy. Typically, a SERS substrate would be provided initially, and the collected and/or captured pathogens from a (body fluid) would be applied upon the substrate. In this way, the (body fluid) sample background will interact/interfere with the SERS substrate, necessitating time-consuming sample pre-processing or accepting poor pathogen signal. In contrast, by providing a nanoparticle coating after the (body) fluid sample is filtered, the sample background has been removed, preventing interaction with the nanoparticles. Therefore, the method is in particular suitable for direct testing of fluid sample, and in particular body fluid samples. However, it is conceivable that preparation steps, such as prefiltration and/or dilution of the sample, are done prior to the nanoparticle coating steps. It is also not excluded to apply the method according to the present invention in combination with a cultivated sample.

Further, since the method steps are relatively simple, there is no trained of qualified professional needed to perform the preparation of the SERS substrate. The method could be performed in a manual way. However, it is not excluded that at least part of the method could be performed in an automated manner. Yet another benefit of the method according to the present invention is that the methods only produces a relatively small amount of waste. Depending on the applied sample, typically the liquid waste produced after the method is sterile or substantially sterile and could therefore possibly be discharged via the conventional routes.

Preferably, the sample is filtered or flushed through at least one coarse filter prior to the sample being filtered through the catch filter. Said coarse filter, if applied, is in particular configured to remove at least a fraction of particles from the sample, in particular particles which are larger than at least one analyte, for example pathogens. This is beneficial as the analytes, such as pathogen could be isolated and/or captured more efficiently. Within the context of this invention, when it is referred to filtering, flushing fluid through a filter and/or drawing fluid up though a filter can be meant. Within the context of this invention, filtering could also be explained as depositing of a fluid onto at least one filter. It is for example imaginable that at least a fraction of the fluid which is to be filtered is absorbed by at least one filter. Hence, in a possible embodiment it is imaginable that at least one fluid, for example at least one solution comprising nanoparticles, is deposited onto at least one filter, in particular such that at least part of the filter, in particular the catch filter, will be coated with nanoparticles thereby forming a SERS substrate.

When it is referred to a sample or a fluid sample, this could for example be a body fluid sample, more in particular a human and/or animal body fluid sample or a biological fluid, such as but not limited to saliva, blood, blood plasma, urine, sweat, tears, cerebrospinal fluid, lymph fluid, synovial fluid, milk, amniotic fluid and/or derivatives thereof. Alternatively, the sample could be any further fluid that could contain at least one analyte, such as pathogens. Further non-limiting examples of fluid samples are water and/or solutions in the lab. The sample can be a pure sample. However, it is also possible that the sample is diluted and/or dissolved in a solvent. The pathogen could for example be bacteria and/or viruses. Further possible applications for the method and device according to the present invention can be found in the medical sector, agricultural sector, water sector and/or food sector and/or in diagnostics as such. Hence, the method according to the present invention can for example be applied for food testing, drugs testing and/or water analysis. At least one analyte could also be referred to as a target analyte. At least one analyte could for example be a biomolecule, biomarker and/or bio-organism. At least one analyte can for example be a biomolecule, such as lipids, fatty acids, glycolipids, sterols, monosaccharides, vitamins, nutrients, hormones, enzymes, proteins, nucleic acid, a molecule of biological source, neurotransmitters and/or metabolites. It is also imaginable that at least one analyte can be a drugs molecule, such as ciprofloxacin, amoxicillin, cocaine, heroin, dopamine, serotonin, ibuprofen, diclofenac and/or erythropoietin. The method and/or device according to the present invention could for example be applied for doping testing, for example via urine test, for airport custom checks and/or for quality control in medicine production and/or the food industry and/or for contamination monitoring for example in (bio) reactors. At least one analyte is preferably a molecule configured to absorb and/or emit light in particular for use in SERS applications.

The solution comprising nanoparticles is preferably an aqueous solution. It could also be referred to the solution comprising nanoparticles according to the invention as a nanoparticle solution. It is beneficial to use a(n) aqueous solution comprising nanoparticles for the last filtration step in the method which basically functions as a wet coating step. This step of applying the nanoparticles via a (wet) solution is preferred over a dry application method, as the wet processing provides a more stable formation of the layer or coating of nanoparticles. Subjecting the catch filter to air could negatively affect the reproducibility. Hence, it is a benefit of the method according to the invention that the method is highly reproducible.

The method according to the present invention could also be referred to as a method for pathogen filtration and/or analysis. The catch filter according to the present invention could also be referred to as membrane filter, in particular a pathogen specific membrane filter. The catch filter according to the invention could also be referred to as depth filter or 3D mesh filter.

It is imaginable that at least two method steps are subsequent method steps. The method steps are preferably subsequent steps. In particular the order wherein the analyte filtering step, or pathogen filtering step is prior to the catch filter itself being formed into a SERS substrate is beneficial for a successful method according to the present invention. It is however imaginable that an assembly of at least one coarse filter and at least one catch filter and that at least one fluid sample is fluid substantially simultaneously through the filter assembly. At least one solution comprising nanoparticles can subsequently be filtered through at least the catch filer. It is for example imaginable that at least one catch filter and/or at least one coarse filter comprises a filter gradient. The use of a filter comprising a filter gradient can positively contribute to an efficient and effective filter process.

In a further possible embodiment, it is imaginable that at least two filtering steps are performed simultaneously. It is for example imaginable that at least one fluid sample and at least one solution comprising nanoparticles are filtered through at least one filter, in particular at least one catch filer, substantially simultaneously. It is for example imaginable that at least one fluid sample and at least one solution comprising nanoparticles are provided in a mixture. It is also imaginable that at least one fluid sample and at least one solution comprising nanoparticles are filtered through an assembly of filters, preferably comprising at least one coarse filter and at least one catch filter. In yet another embodiment, it is imaginable that at least one fluid sample is filtered through at least two filters substantially simultaneously, and preferably through a filter assembly comprising at least one coarse filter and at least one catch filter and that at least one solution comprising nanoparticles is filtered through at least one filter, in particular said catch filter, subsequently.

At least part of the nanoparticles are preferably metal nanoparticles. For example, at least a fraction of the nanoparticles can be metal nanoparticles. It is also conceivable that substantially all nanoparticles are metal nanoparticles. Preferably at least part of the nanoparticles are noble metal nanoparticles. Metal nanoparticles, and in particular noble metal nanoparticles were found to be rather efficient for use in Raman spectroscopy. It is for example conceivable that at least part of the nanoparticles is selected from the group of silver, nickel, aluminium, gold, platinum, palladium, titanium, copper, cobalt, zinc, and/or combinations thereof. It is for example also conceivable that at least part of the nanoparticles comprises an alloy, in particular of any of the listed preferred metals. In a preferred embodiment, at least part of the nanoparticles are gold nanoparticles. It is also conceivable that at least part of the nanoparticles comprises a combination of (noble) metal and silica, such as silica shelled (metal) nanoparticles. It is also imaginable that at least part of the nanoparticles are metal nanoparticles comprising a porous silica shell.

In a further preferred embodiment, at least part of the nanoparticles are charged nanoparticles. It is for example conceivable that at least part of the nanoparticles are positively charged nanoparticles and/or that at least part of the nanoparticles are negatively charged nanoparticles. Hence, it is conceivable that at least part of the nanoparticles of the solution applied are positively charged nanoparticles. This is in particular beneficial in case analysis of negatively charged pathogens, such as bacteria, is desirable. It was experimentally found that the positively charged nanoparticles tend to provide relatively good coating properties in combination with negatively charged pathogen as captured in the catch filter. For similar reasons, it is also beneficial if at least part of the catch filter is negatively charged. On the other hand, in case the analyte is positively charged, it could be beneficial to apply negatively charged nanoparticles. Additionally, at least part of the catch filter could be positively charged. It is also imaginable that at least part of the nanoparticles is uncharged and/or neutral.

At least part of the nanoparticles could for example be substantially spherical nanoparticles. However, it is also conceivable that at least part of the nanoparticles has a non-spherical configuration. At least part of the nanoparticles could for example be substantially star-, rod-, wire-, oval-, prism-, urchin-, cube-, raspberry- and/or disc shaped. It is also conceivable that at least part of the nanoparticles has a non-uniform configuration. In a preferred embodiment, at least part of the nanoparticles are nanostars and/or nanorods.

In a possible embodiment, at least part of the nanoparticles can be coated nanoparticles. It is for example imaginable that at least part of the nanoparticles can be coated with a metal coating. The coating can for example be a functional coating. Non-limiting example of coatings which could be applied titanium dioxide, silicon dioxide, carbon black, iron oxide, zinc oxide and/or silver. It is also imaginable that at least one coating applied is a non-metal material. At least one coating can for example comprise citrate, thioglucose and/or cetrimonium bromide (CTAB).

Typically, the nanoparticles of at least one solution comprising nanoparticles have an average diameter in the range of 1 to 1000 nm. In a preferred embodiment, at least part of the nanoparticles has an average diameter smaller than 1000 nm, preferably smaller than 500 nm, more preferably smaller than 100 nm, and even more preferably smaller than 60 nm. At least part of the nanoparticles can for example have an average diameter in the range of 20 nm to 100 nm, preferably in the range of 40 nm to 60 nm, more preferably in the range of 45 nm to 55 nm. It is also conceivable that at least part of the nanoparticles can for example have an average diameter in the range of 50 nm to 800 nm, preferably in the range of 100 nm to 600 nm, more preferably in the range of 200 nm to 400 nm. It is also conceivable that at least part of the nanoparticles has an average width and/or length in the range of 20 nm to 100 nm, preferably in the range of 40 nm to 60 nm, more preferably in the range of 45 nm to 55 nm. It is also conceivable that at least part of the nanoparticles has an average width and/or length smaller than 1000 nm, preferably smaller than 500 nm, more preferably smaller than 100 nm, and even more preferably smaller than 60 nm.

The method could further comprise the step of flushing at least the catch filter with a washing solution prior to said catch filter is flushed with the solution comprising nanoparticles. It is for example conceivable that at least the catch filter is flushed with a washing solution comprising at least one salt and/or a (buffered) saline solution. It is also conceivable that at least the catch filter is flushed with at least one washing solution comprising at least one salt and/or at least one detergent. The washing step could for example remove pigments and/or further contaminants from the catch filter. It is also conceivable that the washing step is applied to further optimize the overall process. It is conceivable that multiple washing steps are applied in the method according to the present invention.

It is conceivable that at least one coarse filter comprises at least one coarse-grained fabric material, such as but not limited to: glass wool, cotton, synthetic wool and the like. In a beneficial embodiment, at least one coarse filter, if applied, comprises glass fiber and/or glass wool. It is for example possible that at least one coarse filter is formed by glass wool comprising or being formed by glass fiber. Glass wool and glass fiber were found to be efficient in a filtering step wherein relatively large particles are to be separated from a sample, in particular a body fluid sample. The use of glass wool and/or glass is also beneficial from economical point of view since said products are relatively cheap. It is also conceivable that at least one coarse filter comprises cellulose. Other non-limitative embodiments include at least one coarse filter comprising polycarbonate and/or a coarse filter which is at least partially made of a filter paper, coffee filter or coffee paper. It is also conceivable that at least one coarse filter as applied in a method according to the present invention comprising multiple filtering materials. Hence, the coarse filter could for example be a combination of glass wool and cellulose. It is further imaginable that at least one coarse filter comprises glass fiber and/or wherein at least one catch filter comprises a glass fiber mesh, at least one hydrophilic polymer, glass wool, nylon, polyamide, cellulose acetate, polysulphone, teflon and/or cellulose. At least one coarse filter could further comprise at least one material chosen from the group of: Polycarbonate (PC), Polyvinylidene fluoride (PVDF), NylonCellulose NitrateMixed Cellulose Ester (MCE), Anodized Aluminum Oxide (AAO), Polyethersulfone (PES), Polytetrafluoroethylene (PTFE), Polypropylene (PP), Glass Fiber Filter (GMF), Ceramic Membranes, Track-Etched Membrane Filters, Silica-based Membranes, Carbon Nanotube Membranes, Polyamide (POA), Cellulose acetate (CEA)Polycarbonate (POC), Polypropylene (PP), Cellulose S/G (hydrophilic polymers, coated and uncoated), Polysulfone (PS) and/or Polyethersulfone (PES).

At least one coarse filter is configured to capture particles with a particle size larger than 10 μm, preferably larger than 7 μm, more preferably larger than 6 μm, even more preferably larger than 5 μm or larger than 2 μm or 1 μm. It is also conceivable that at least one coarse filter is configured to capture particles with a particle size (significantly) larger than 8 μm. At least on coarse filter is in particular configured to let through, particles with a particle size smaller than 10 μm or 7 μm, preferably smaller than 6 μm, more preferably smaller than 5 μm. In this manner, any pathogens present in the sample, which are typically smaller than said particle size, will be let through towards the catch filter step while larger contaminants will be removed from the sample. When it is referred to particle size, for example an average particle size can be meant. It is also conceivable that one coarse filter is configured to capture particles with a particle size in the range of 5 μm to 10 μm, preferably 6 μm to 8 μm. It is also conceivable that at least one coarse filter is configured to capture particles with an average width and/or length in the range of 5 μm to 10 μm.

At least one coarse filter, if applied, could possibly be configured to filter at least part of white blood cells, red blood cells, epithelial cells and/or (salt) crystals from the sample, in particular the body fluid sample. Hence, at least one coarse filter can be configured to capture at least part of white blood cells, red blood cells, epithelial cells and/or (salt) crystals in said filter. It is also imaginable that at least one coarse filter is configured to filter at least part of the molecules larger than at least one analyte from the fluid sample. The use of at least one coarse filter can also be useful when applying the method and/or device according to the present invention in water, food and/or agriculture applications. It is for example imaginable that at least one coarse filter is used to filter sediment, suspended solids, insects, organisms and/or debris. It is also imaginable that at least one coarse filter is configured to filter relatively large particulate matter such as insects and pests, for example oversized foreign objects. It is also conceivable that a combination of coarse filters is applied to filter one or more of said target particles. It is beneficial to apply a coarse filter, as this could further optimize the SERS substrate formation. Particles larger than pathogen could negatively affect the Raman spectroscopy if they would still be present upon the SERS substrate. It is also conceivable that the method comprises the step of flushing or filtering the sample, in particular the body fluid sample, through at least two coarse filters, wherein a first coarse filter is configured to remove at least a fraction of particles with a first predetermined particle size or first particular size range and wherein a second coarse filter is configured to remove at least a fraction of particles with a second predetermined particle size or second particle size range. Hence, in this manner, contamination particles could be removed from the sample in a more controlled manner. It is also conceivable that a (first) coarse filters prevents clogging at least one further filter, such as a second coarse filter and/or the catch filter. It is for example imaginable that particles with a first particle size are epithelial cells, white blood cells and/or (salt) crystals and particles having a second particle size are red blood cells. It is also conceivable that a coarse filter as applied for the method according to the present invention is an assembly of multiple individual coarse filters or coarse filter membranes. Coarse filtration could alternatively also be realized by using a resin as applied in size exclusion chromatography. It also conceivable that at least one coarse filter is applied in an assembly in combination with at least one catch filter. In such configuration, the sample could preferably be flushed through the filter assembly in a single step, in particular wherein the coarse filter is configured to filter the contamination particles prior to the capturing of the pathogen by the catch filter. If an assembly of at least one coarse filter and at least one catch filter is applied, it is also conceivable that the method comprises the step of removal of at least one coarse filter from the assembly prior to the nanoparticle coating step.

At least one catch filter comprises glass in a preferred embodiment. It is also conceivable that at least one catch filter is at least partially made of glass. The catch filter could for example comprise glass particles, for example glass nanoparticles. At least one catch filter could for example comprise a glass (fiber) mesh. It is beneficial if the catch filter comprises a mesh structure or configuration, as this positively contributes the formation of a randomized three-dimensional pattern of nanoparticles and captured pathogens. This will positively contribute to any further pathogen analysis steps, for example in Raman spectroscopy. The catch filter is preferably a relatively dense filter. At least part of the catch filter could for example be coated. It is also conceivable that the method comprises a step wherein the at least part of the catch filter is coated. Coating could for example be done via a wet coating process, for example but not limited to the use of a positively charged CTAB detergent coating or antibody coating. It is further imaginable that at least one catch filter comprises glass fiber and/or wherein at least one catch filter comprises a glass fiber mesh, glass wool, nylon, polyamide, cellulose acetate, polysulphone, teflon and/or cellulose. It is imaginable that at least one filter is substantially hydrophobic. Preferably, at least part of at least one catch filter is substantially hydrophobic. It is for example conceivable that at least one catch filter comprises at least one hydrophilic polymer. At least one catch filter could further comprise at least one material chosen from the group of: Polycarbonate (PC), Polyvinylidene fluoride (PVDF), NylonCellulose NitrateMixed Cellulose Ester (MCE), Anodized Aluminum Oxide (AAO), Polyethersulfone (PES), Polytetrafluoroethylene (PTFE), Polypropylene (PP), Glass Fiber Filter (GMF), Ceramic Membranes, Track-Etched Membrane Filters, Silica-based Membranes, Carbon Nanotube Membranes, Polyamide (POA), Cellulose acetate (CEA)Polycarbonate (POC), Polypropylene (PP), Cellulose S/G (hydrophilic polymers, coated and uncoated), Polysulfone (PS) and/or Polyethersulfone (PES).

At least one catch filter is preferably configured to capture particles with a particle size smaller than 5 μm, preferably smaller than 3 μm, more preferably smaller than 2.5 μm, even more preferably smaller than 2 μm and most preferably smaller than 0.5 μm. In yet a further preferred embodiment, at least one catch filter is configured to capture particles with a particle size smaller than 1 μm, more in particular smaller than 0.7 μm, more in particular smaller than 0.4 μm. It may also be said that the catch filter is configured to catch particles with a predetermined particle size, for example in any of the abovementioned ranges and/or any of the ranges mentioned for the (target) analyte.

In a preferred embodiment, at least one coarse filter and/or at least one catch filter can be present in a syringe filter. It is for example also imaginable that at least one coarse filter and/or at least one catch filter are comprised in a syringe filter. At least one coarse filter and/or at least one catch filter could for example also be be a syringe filter. The filtering step could for example be applied by pushing the sample across at least one filter by the syringe. The use of at least one syringe filter in combination with a syringe could enable manual application of the method in a relatively simple manner. It is also conceivable that at least one coarse filter and/or at least one catch filter is present in a syringe filter device. Preferably, at least one filter is provided with a housing. The use of a housing could further contribute to the ease of the use of the filter. Optionally or alternatively, a filter applied within the scope of this invention could be a drip filter.

In a preferred embodiment, the method comprises the step of at least partially drying the catch filter after said catch filter is flushed with the solution comprising nanoparticles. At least partially drying the catch filter could positively contribute to the use of the filter in a Raman spectroscopy process. It is for example conceivable that the drying step is performed during a predetermined amount of time. Typically, at least partially drying of the catch filter could take several minutes.

The method could further include the step of using the nanoparticle coated catch filter (as a SERS substrate) in a Raman spectroscopy analysis. Raman spectroscopy could be applied in order to identify, quantify and/or classify the presence of any pathogens. The Raman spectroscopy analysis in a method according to the present invention could for example be applied by making use of at least one handheld Raman spectroscopy device, a handheld and/or portable spectrometer and/or a desktop spectrometer.

The invention also relates to a method for analysing the presence of at least one analyte in a fluid sample, comprising the steps of preparing a SERS substrate for analysis of at least one analyte in a fluid sample according to the present invention and the method comprising the step of applying the nanoparticle coated catch filter as SERS substrate in a Raman spectroscopy step in order to analyse the presence of at least one analyte. The invention also relates to a method for analysing the presence of pathogens in a body fluid sample, comprising the steps of preparing a SERS substrate for pathogen analysis in a body fluid sample according to the present invention and the method comprising the step of applying the nanoparticle coated catch filter as SERS substrate in a Raman spectroscopy step in order to analyse the presence of pathogen.

In fact, the invention also relates to a method for analysing the presence of pathogens in a sample, in particular a body fluid sample, comprising the steps of providing at least one sample, in particular a body fluid sample, providing at least one filter assembly, wherein the filter assembly comprises optionally at least one coarse filter configured to remove at least a fraction of particles from the sample, in particular the body fluid sample, in particular particles larger than pathogens, and at least one catch filter configured to capture at least pathogens if present in the sample, in particular the body fluid sample, flushing or filtering the sample, in particular the body fluid sample through the filter assembly, flushing or filtering at least one (aqueous) solution comprising nanoparticles through at least the catch filter such that at least part of the catch filter will be (covered and/or) coated with nanoparticles, and applying the nanoparticle coated catch filter as SERS substrate in a Raman spectroscopy step in order to analyse the presence of pathogens. This method could be applied in combination with any of the previously described embodiments.

The invention further relates to the use of at least one nanoparticle coated catch filter as SERS substrate in a Raman spectroscopy analysis and/or to at least one nanoparticle coated catch filter configured for use as SERS substrate in a Raman spectroscopy analysis. The nanoparticles could be any of the nanoparticles as described for the present invention. The catch filter could be any of the described embodiments of catch filters according to the present invention.

The invention also relates to a device for preparing a SERS substrate for analysis of at least one analyte in a fluid sample, for example pathogen analysis in a body fluid sample, in particular for use in a method according to the present invention, the device comprising:
at least one filter assembly, wherein the filter assembly comprises:
   optionally at least one coarse filter configured to remove at least a fraction of particles from the fluid sample wherein the particles to be removed are larger than at least one analyte,, and
   at least one catch filter configured to capture at least a fraction of at least one analyte, such as pathogens, from the fluid sample.

The device according to the present invention could be applied in combination with a method according to the present invention. The device also enables the application of a method according to the present invention, in particular in a practical manner. It is for example imaginable that at least one coarse filter is configured to remove at least a fraction of particles from the body fluid sample, in particular particles larger than pathogens.

The filter assembly preferably has a substantially modular configuration. It is for example conceivable that at least one coarse filter and at least one catch filter are mutually connected and/or connectable in a releasable manner. This enables that only a single filtering step of the (body) fluid sample can be done, saving time and effort, whilst the catch filter can be easily prepared subsequently into a SERS substrate for Raman spectroscopy.

At least one coarse filter and at least one catch filter can be any of the described embodiments as could be applied in the method according to the present invention. It is for example conceivable that at least one coarse filter comprises glass fiber, glass wool and/or cellulose. At least one coarse filter could also comprise at least one glass fiber mesh glass, glass wool, nylon, a hydrophilic polymer, polyamide, cellulose acetate, polysulphone, teflon and/or cellulose. It is also imaginable that at least one coarse filter is configured to capture particles with a particle size larger than 7 μm, preferably larger than 6 μm, more preferably larger than 5 μm. The coarse filter could for example be configured to let through particles with a particle size smaller than 7 μm, preferably smaller than 6 μm, more preferably smaller than 5 μm. At least one catch filter could for example comprise glass, in particular a glass fiber mesh, glass wool, nylon, polyamide, cellulose acetate, polysulphone, teflon, cellulose and/or at least one hydrophilic polymer. At least one catch filter is preferably configured to capture particles with a particle size smaller than 5 μm, preferably smaller 3 μm, more preferably smaller than 2.5 μm, most preferably smaller than 2 μm.

It is also imaginable that at least one catch filter and/or at least one coarse filter comprises at least one material chosen from the group of: Polycarbonate (PC), Polyvinylidene fluoride (PVDF), NylonCellulose NitrateMixed Cellulose Ester (MCE), Anodized Aluminum Oxide (AAO), Polyethersulfone (PES), Polytetrafluoroethylene (PTFE), Polypropylene (PP), Glass Fiber Filter (GMF), Ceramic Membranes, Track-Etched Membrane Filters, Silica-based Membranes, Carbon Nanotube Membranes, Polyamide (POA), Cellulose acetate (CEA)Polycarbonate (POC), Polypropylene (PP), Cellulose, S/G (hydrophilic polymers, coated and uncoated), Polysulfone (PS) and/or Polyethersulfone (PES). It is for example also conceivable that at least one coarse filter comprises at least one hydrophilic polymer.

In a preferred embodiment of the filter assembly, at least one coarse filter and/or at least one catch filter is a syringe filter and/or is comprised in a syringe filter. In this way, the sample could be filtered through the filters in a relatively simple manner. The sample, in particular a body fluid sample, could be provided in a syringe which could be connected to the filter assembly. Hence, the filter assembly could be configured for co-action with a syringe. It is also conceivable that at least one coarse filter is received within a housing and/or that at least one catch filter is received within a housing. The presence of a housing could enables easy handling of the catch filter and/or coarse filter. In a practical embodiment, at least one housing comprises at least one catch filter and/or at least one coarse filter. Such housing may comprise at least one receiving space for receiving at least one filter.

It is also imaginable that the device and in particular at least one catch filter and/or at least one coarse filter comprises a detachable lid. It is for example imaginable that the device, and in particular at least one catch filter, is configured for co-action with at least one filter lid and/or at least one scanner lid. The use of a filter lid and/or scanner lid can further optimize the efficiency and ease of use of the method and device according to the present invention. At least one filter lid can for example be configured to facilitate a connection with a syringe which is typically used for filtering the sample fluid and/or the solution comprising nanoparticles or optionally a washing fluid. At least one scanner lid can be configured to facilitate a connection with a Raman spectroscopy device or an analysis aid thereof, for example a housing.

It is also conceivable that the device according to the present invention comprises at least one gripping structure in particular for manual gripping of the device. This could be beneficial from safety point of view as the gripping structure could for example be configured to prevent a user from getting into (direct) contact with the actual (liquid) sample or nanoparticle solution. Hence, this is beneficial from hygienic point of view. In a possible embodiment, at least one gripping structure at least partially encloses the filter assembly. In this way, the co-action between the gripping structure and the filter assembly could be further optimized. Optionally, at least one gripping structure comprises a plurality of substantially flexible gripping members. At least one gripping structure, if applied, could be connected or connectable to the housing of a filter or the filter assembly. The gripping structure could in a beneficial embodiment comprise at least two protruding gripper members, preferably at least three protruding gripping members. Such gripping members could positively contribute to the ease of use of the device and/or the stability of the device as such. The use of at least one gripping structure could for example positively contribute to centrical mounting of at least the filter assembly, or the device as such, within a container, such as a beaker. This could ensure decent waste disposal and vertical positioning of device while this setup arranges clamping of the part which houses the SERS-substrate during process and eases the extraction of this part after lifting the device out of the beaker while design avoids (processed) sample being spilled.

Possibly, the device could comprise at least one fluid distribution structure for dividing fluid over the filter. It is for example possible that the fluid distribution structure forms part of at least one filter, e.g. the catch filter and/or coarse filter. It is also possible that at least one distribution structure forms (integral) part of a housing of a filter or of the filter assembly. The fluid distribution structure could for example comprise a raster configuration. It is also conceivable that the device comprises at least one guiding surface, in particular for guiding filtered (body fluid) sample away from the filter(s), in particular the catch and/or coarse filter. At least one guiding surface could for example be provided in an internal volume of the filter assembly. It is also possible that at least one catch filter and/or at least one coarse filter comprises at least one guiding surface. It is for example also conceivable that the filter device, the filter assembly, at least one catch filter and/or at least one coarse filter, comprises a double wall configuration. Such configuration could also positively contribute to the ease of use of the device and could be beneficial from hygienic point of view.

The invention will be further elucidated based upon the following non-limitative clauses.

1. Method for preparing a SERS substrate for pathogen analysis in a body fluid sample, comprising the steps of:
providing at least one body fluid sample;
optionally filtering the body fluid sample through at least one coarse filter, wherein the coarse filter is configured to remove at least a fraction of particles from the body fluid sample, in particular particles which are larger than pathogens;
filtering the body fluid sample through at least one catch filter, wherein the catch filter is configured to capture pathogens if present in the body fluid sample;
filtering at least one solution comprising nanoparticles through the catch filter such that at least part of the catch filter will be coated with nanoparticles thereby forming a SERS substrate.

2. Method according to clause 1, wherein the steps are subsequent steps.

3. Method according to any of the previous clauses, wherein at least part of the nanoparticles are metal nanoparticles.

4. Method according to any of the previous clauses, wherein at least part of the nanoparticles is selected from the group of silver, nickel, aluminium, gold, platinum, palladium, titanium, copper, cobalt, zinc, and/or combinations thereof.

5. Method according to any of the previous clauses, wherein at least part of the nanoparticles are positively charged nanoparticles.

6. Method according to any of the previous clauses, wherein at least part of the nanoparticles are substantially spherical nanoparticles.

7. Method according to any of the previous clauses, wherein at least part of the nanoparticles has an average diameter in the range of 20 nm to 100 nm, preferably in the range of 40 nm to 60 nm, more preferably in the range of 45 nm to 55 nm.

8. Method according to any of the previous clauses, comprising the step of flushing at least the catch filter with a washing solution, preferably a washing solution comprising at least one salt, prior to said catch filter is flushed with the solution comprising nanoparticles.

9. Method according to any of the previous clauses, wherein at least one coarse filter comprises glass fiber and/or glass wool.

10. Method according to any of the previous clauses, wherein at least one coarse filter comprises cellulose.

11. Method according to any of the previous clauses, wherein at least one coarse filter is configured to capture particles with a particle size larger than 7 μm, preferably larger than 6 μm, more preferably larger than 5 μm.

12. Method according to any of the previous clauses, wherein at least one coarse filter is configured to filter at least part of white blood cells, red blood cells, epithelial cells and/or crystals from the body fluid sample.

13. Method according to any of the previous clauses, comprising the step of flushing the body fluid sample through at least two coarse filters, wherein a first coarse filter is configured to remove at least a fraction of particles with a first predetermined particle size and wherein a second coarse filter is configured to remove at least a fraction of particles with a second predetermined particle size.

14. Method according to any of the previous clauses, wherein at least one catch filter comprises glass, in particular glass fiber.

15. Method according to any of the previous clauses, wherein at least one catch filter comprises a glass fiber mesh.

16. Method according to any of the previous clauses, wherein at least one catch filter is configured to capture particles with a particle size smaller than 5 μm, preferably smaller than 3 μm, more preferably smaller than 2.5 μm, most preferably smaller than 2 μm.

17. Method according to any of the previous clauses, wherein at least one coarse filter and/or at least one catch filter is a syringe filter.

18. Method according to any of the previous clauses, comprising the step of at least partially drying the catch filter after said catch filter is flushed with the solution comprising nanoparticles.

19. Method according to any of the previous clauses, comprising the step of using the nanoparticle coated catch filter in a Raman spectroscopy analysis.

20. Method for pathogen analysis in a body fluid sample, comprising the steps of preparing a SERS substrate for pathogen analysis in a body fluid sample according to any of the previous clauses and comprising the step of applying the nanoparticle coated catch filter as SERS substrate in a Raman spectroscopy analysis in order to analyse and/or identify any pathogens present.

21. Use of at least one nanoparticle coated catch filter as SERS substrate in a Raman spectroscopy step.

22. Device for preparing a SERS substrate for pathogen analysis in a body fluid sample, in particular for use in a method according to any of clauses 1 to 20, comprising:
at least one filter assembly, wherein the filter assembly comprises:
   at least one coarse filter configured to remove at least a fraction of particles from the body fluid sample, in particular particles larger than pathogens; and
   at least one catch filter configured to capture at least pathogens from the body fluid sample.

23. Device according to clause 22, wherein at least one coarse filter and at least one catch filter are mutually connectable in a releasable manner.

24. Device according to clause 22 or clause 23, wherein at least one coarse filter comprises glass fiber, glass wool and/or cellulose.

25. Device according to any of clauses 22 to 24, wherein at least one coarse filter is configured to capture particles with a particle size larger than 7 μm, preferably larger than 6 μm, more preferably larger than 5 μm.

26. Device according to any of clauses 22 to 25, wherein at least one catch filter comprises glass, in particular a glass mesh.

27. Device according to any of clauses 22 to 26, wherein at least one catch filter is configured to filter particles with a particle size smaller than 3 μm, preferably smaller than 2.5 μm, more preferably smaller than 2 μm.

28. Device according to any of clauses 22 to 27, wherein at least one coarse filter and/or at least one catch filter is a syringe filter.

29. Device according to any of clauses 22 to 28, wherein at least one coarse filter is received within a housing and/or wherein at least one catch filter is received within a housing.

30. Device according to any of clauses 22 to 29, comprising at least one gripping structure for manual gripping of the device.

31. Device according to clause 30, wherein at least one gripping structure comprises a plurality of substantially flexible gripping members.

32. Device according to any of clauses 22 to 31, comprising at least one fluid distribution structure for dividing fluid over the filter.

33. Device according to any of clauses 22 to 32, comprising at least one guiding surface in particular for guiding filtered sample away from at least one filter.

The invention will be further elucidated by means of non-limiting exemplary embodiments illustrated in the following figures, in which:

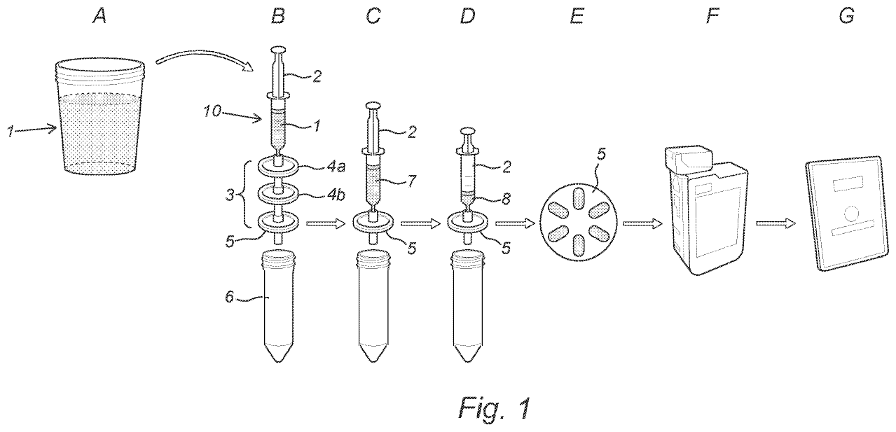

FIG. 1 shows a sequence of steps of a method according to the present invention;

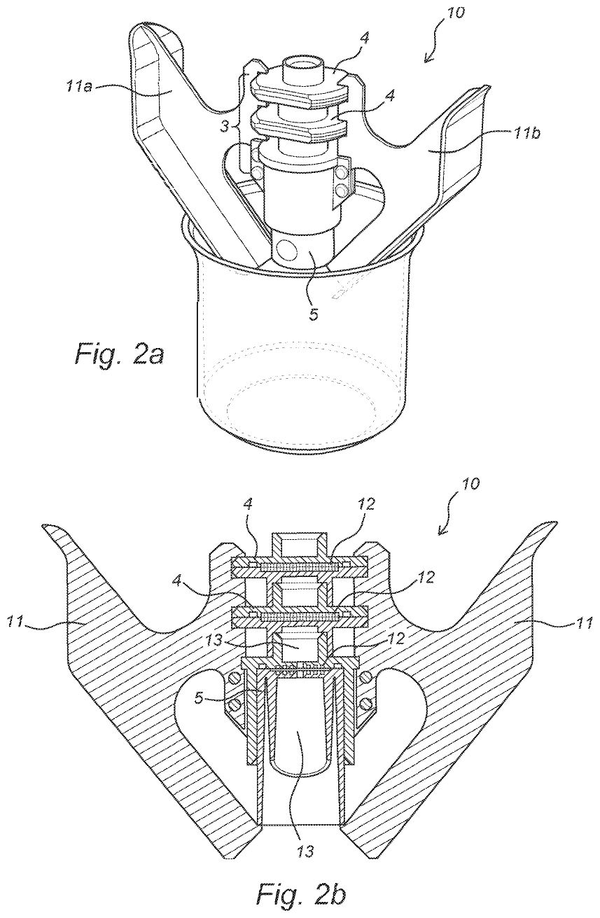

FIGS. 2a and 2b show a further possible embodiment of a device according to the present invention;

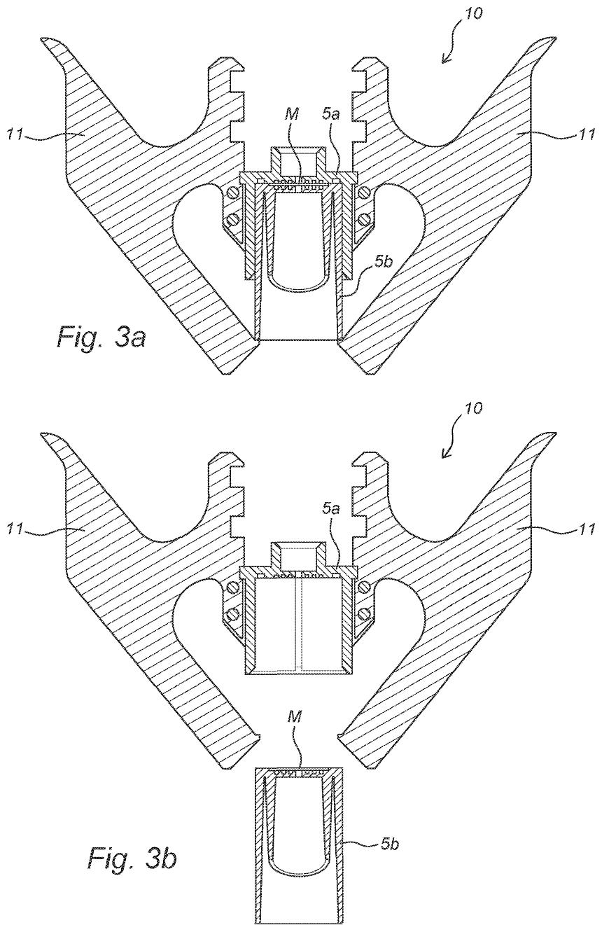

FIGS. 3a and 3b show part of the device as shown in FIGS. 2a and 2b;

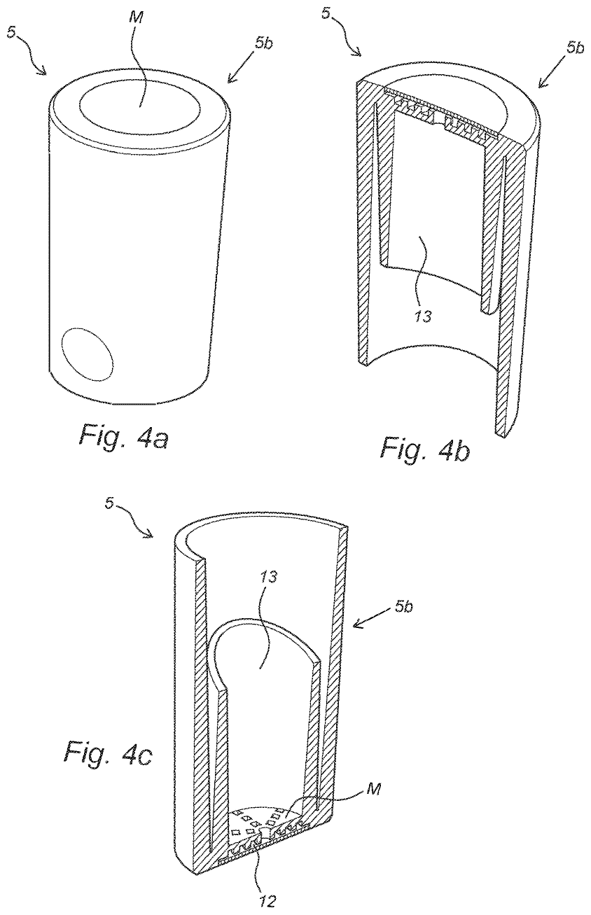

FIGS. 4a, 4b and 4c shown part of the catch filter as applied in the device of FIGS. 2a-3b; and

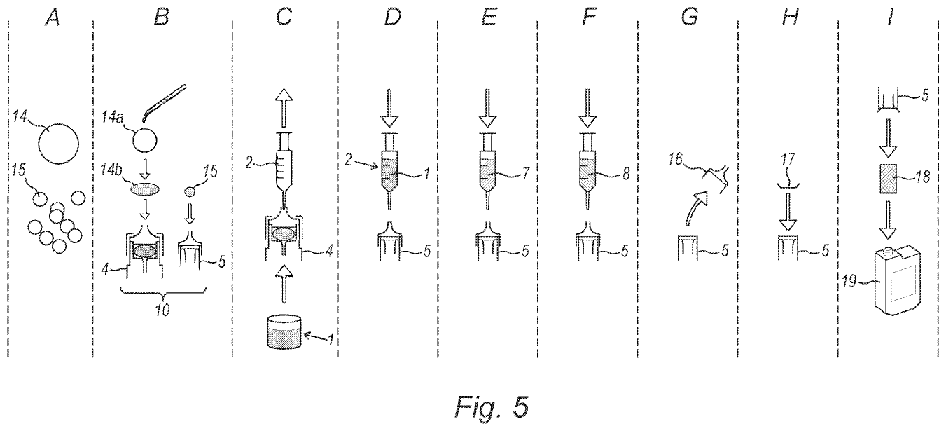

FIG. 5 shows a further example of a sequence of steps of a method according to the present invention.

Within these figures, similar reference numbers correspond to similar or equivalent elements or features.

FIG. 1 shows a sequence of steps of a method according to the present invention. The figure shows the preparation of a SERS substrate for pathogen detection, pathogen identification and/or pathogen analysis in a sample, in particular a body fluid sample and the subsequent (optional) analysing steps in order to verify if pathogens such as bacteria were present in the sample. The figure shows a sequence of steps A to G making use of a device 10 according to the present invention. Step A shows the provision of a body fluid sample 1. This could be any body fluid wherein pathogen such as bacteria could be present, such as but not limited to blood, blood plasma, urine, sweat, tears, cerebrospinal fluid, lymph fluid, synovial fluid, milk, amniotic fluid and/or derivatives thereof. In the shown embodiment of the method, the body fluid sample 1 is drawn up in a syringe 2. The syringe 2 is then connected to a filter assembly 3. In the shown embodiment, the filter assembly 3 comprises two coarse filters 4a, 4b and a catch filter 5. The coarse filters 4a, 4b are configured to remove at least a fraction of particles from the body fluid sample 1 which are larger than pathogens, in particular bacteria. The catch filter 5 is configured to capture pathogens if present in the body fluid sample 1. Step B shows that the body fluid sample 1 is flushed through the coarse filters 4a, 4b and through the catch filter 5. In the shown embodiment, the waste fluid 6 is collected in a container 6, in particular a vial 6. After step B, the coarse filters 4a, 4b are removed from the filter assembly 3. Step C is optional, and shows the step of flushing the catch filter 5 with a washing solution 7, in particular a washing solution comprising at least one salt and/or at least one detergent. This step could for example remove pigment(s) from the catch filter 5. Step D shows the flushing of the catch filter 5 with a solution comprising nanoparticles 8. During this step, at least part of the catch filter 5 will be coated with nanoparticles 8 such that a substrate is formed which can be applied as SERS substrate. The nanoparticles 8 are preferably metal nanoparticles 8, more preferably gold nanoparticles 8. The filter 5 as shown in step D can be seen as a substrate which is formed by the filter material of the catch filter 5 and nanoparticles, wherein any captured pathogens are entrapped and/or embedded within the filter material and the nanoparticles. Step E shows that the nanoparticle coated catch filter 5 is applied as SERS substrate in a Raman spectroscopy analysis. Further detailed analysis of the spectroscopy results, for example pathogen analysis can be done in step G. The presence and/or identification of pathogens is enabled by this method in a relatively simple, fast and efficient manner. The shown steps are in particular subsequent steps. In the shown embodiment, the coarse filters 4a, 4b and the catch filter 5 are syringe filters. This enables easy connection of the syringe 2 to the filter assembly 3 or filters 4a, 4b, 5 as such.

FIGS. 2a and 2b show a further possible embodiment of a device 10 according to the present invention. FIG. 2a shows a perspective view wherein the device 10 is received in a container 6 whereas FIG. 2b shows a cross section of said device 10. The device 10 is in particular configured for preparing a SERS substrate for pathogen analysis in a body fluid sample. The device 10 comprises a filter assembly 3 comprising a coarse filter 4 configured to remove at least a fraction of particles from the body fluid sample, in particular particles larger than pathogens such as bacteria and a catch filter 5 configured to capture at least pathogens such as bacteria. In the shown embodiment, a plurality of separate coarse filters 4 forms the actual coarse filter. The filters 4, 5 have a modular configuration making it easy to connect and/or disconnect several filters 4, 5. This is in particular of benefit to remove the coarse filter(s) 4 from the catch filter 5. Each filter 4, 5 is thereto provided with a housing. The filters 4, 5 are hence stackable to each other and connectable to a fluid supply such as but not limited to a syringe (not shown). The device 10 further comprises a gripping structure 11 for manual gripping of the device 10. In the shown embodiment, the gripping structure 11 at least partially encloses the filter assembly 3. The gripping structure 11 comprises a plurality of substantially flexible gripping members 11a, 11b. In the shown embodiment, the device 10, and in particular each filter 4, 5, comprises a fluid distribution structure 12 for dividing fluid over the filter 4, 5. The filters 4, 5 further comprise a guiding surface 13 for guiding filtered liquids.

FIGS. 3a and 3b show the device 10 as shown in FIGS. 2a and 2b in a configuration wherein the coarse filters are removed from the filter assembly. The figures shows that the catch filter 5 itself also has a modular configuration. The catch filter 5 as shown comprises an upper catch filter part 5a and a lower catch filter part 5b. The actual filter, or filter membrane M, is received within the upper catch filter part 5a and the lower catch filter part 5b. This is practical as the filter can be easily removed from the assembly after the method steps have been performed. It can be seen that the gripping structure 11 is designed such that the lower catch filter part 5b can be substantially retained but also easily and safely removed by actuating gripping members 11a, 11b in the connection configuration of the catch filter parts 5a, 5b.

FIGS. 4a, 4b and 4c show a detailed view of the lower catch filter part 5b as shown in FIGS. 2a-3b. It can be seen that the filter 5, in particular the lower catch filter part 5b, has a double wall configuration. The filter 5 comprises a fluid distribution structure 12 for dividing fluid over the filter 5 and a guiding surface 13 for guiding filtered liquids. It is also conceivable that the catch filter 5, in particular the lower catch filter part 5b, is configured to be applied as housing in a Raman spectroscopy analysis.

FIG. 5 shows a further example of a sequence of steps of a method according to the present invention. The figure shows the preparation of a SERS substrate for analysis of at least one analyte in a fluid sample and the subsequent (optional) analysing steps in order to verify if the analyte was present in the sample. The figure shows a sequence of steps A to I making use of a device 10 according to the present invention. Step A shows the preparation of the filter materials 14, 14 of the coarse filter 4 and the catch filter 5. Step B shows the preparation of the device 10 wherein, in the shown embodiment, the filter materials 14a, 14b used in the coarse filters 4 are comprises in a first syringe filter. The material 15 used in the catch filter 5 is comprised in a second syringe filter. Step C shows the provision of a fluid sample 1. In the shown embodiment of the method, the fluid sample 1 is drawn up in a syringe 2 via the coarse filter 4. Subsequently, step D shows that the filtered fluid sample present in the syringe 2 is flushed though the catch filter 5. The catch filter 5 is configured to capture at least a fraction of the analyte(s) if present in the fluid sample 1. Step E is an optional step wherein a washing solution 7 is flushed through the catch filter 5. This step could for example remove pollutants such as pigment(s) from the catch filter 5. Step F shows the flushing of the catch filter 5 with a solution comprising nanoparticles 8. During this step, at least part of the catch filter 5 will be coated with nanoparticles 8 such that a substrate is formed which can be applied as SERS substrate. Step G shows that a filter lid 16 of the catch filter 5 is removed. Subsequently, a scanner lid 17 is attached to the catch filter 15. The catch filter 5 is then ready to be used in a Raman spectroscopy analyse. Preferably, the catch filter 5 is thereto received in a housing 18 before being positioned in the Raman spectroscopy device 19.

There are a number of possible applications in which the method and device according to the present invention could be applied. Non-limiting examples of possible applications to which the method and/or device according to the present invention could be directed are listed hereinafter: Chemical sensing, for example: Explosives such as: TNT (trinitrotoluene), RDX (hexahydro-1,3,5-trinitro-1,3,5-triazine); Drugs such as: Cocaine, heroin, methamphetamine; Environmental pollutants, for example: Polycyclic aromatic hydrocarbons (PAHs), pesticides, heavy metals; Biomedical applications, for example: Proteins, such as: Albumin, insulin, antibodies; DNA and RNA, such as: Gene sequences, mutations, DNA-binding proteins; Cancer biomarkers, such as: Epidermal growth factor receptor (EGFR), human epidermal growth factor receptor 2 (HER2), prostate-specific antigen (PSA); Metabolites, such as: Glucose, lactate, cholesterol, amino acids; Small molecules, such as: Adenosine triphosphate (ATP), cyclic adenosine monophosphate (cAMP), neurotransmitters (e.g., dopamine, serotonin); Bacteria, such as: Staphylococcus aureus, Escherichia coli (E. coli), Mycobacterium tuberculosis; Viruses, such as: Influenza virus, Human immunodeficiency virus (HIV), Zika virus; Fungi, such as: Candida albicans, Aspergillus fumigatus, Cryptococcus neoformans; Food safety and quality control, for example: Pesticides, such as: Glyphosate, chlorpyrifos, neonicotinoids; Toxins, such as: Aflatoxins, mycotoxins, histamine; Food additives, such as: Preservatives (e.g., benzoic acid, sorbic acid), sweeteners (e.g., aspartame, saccharin), colorants (e.g., tartrazine, carmine); Bacteria, such as: Salmonella, Escherichia coli (E. coli), Listeria monocytogenes; Viruses, such as: Norovirus, Hepatitis A virus, Rotavirus; Parasites, such as: Cryptosporidium, Giardia, Toxoplasma gondii; Forensic analysis, for example: Drugs, such as: Cocaine, heroin, amphetamines; Gunshot residues, such as: Lead, barium, antimony; Fibers, such as: Synthetic fibers (e.g., polyester, nylon), natural fibers (e.g., cotton, wool); Environmental monitoring, for example: Water pollutants, such as: Heavy metals (e.g., mercury, lead), organic compounds (e.g., polychlorinated biphenyls, pharmaceuticals); Air pollutants, such as: Volatile organic compounds (VOCs), particulate matter, nitrogen oxides (NOx); Soil contaminants, such as: Polycyclic aromatic hydrocarbons (PAHs), pesticides, industrial waste byproducts; Pharmaceutical research, for example: Drug molecules, such as: Ibuprofen, paracetamol (acetaminophen), aspirin; Drug delivery systems, such as: Liposomes, micelles, nanoparticles; Active pharmaceutical ingredients (APIs), such as: Antibiotics, antiviral drugs, anticancer agents; Nanotechnology and material science, for example: Nanomaterials, such as: Gold nanoparticles, silver nanoparticles, carbon nanotubes; Thin films, such as: Polymer films, oxide films, graphene layers; Surface properties, such as: Adsorbed molecules, functionalized surfaces, self-assembled monolayers, and/or combination thereof.

It will be clear that the invention is not limited to the exemplary embodiments which are illustrated and described here, but that countless variants are possible within the framework of the attached claims, which will be obvious to the person skilled in the art. In this case, it is conceivable for different inventive concepts and/or technical measures of the above-described variant embodiments to be completely or partly combined without departing from the inventive idea described in the attached claims. Alternatively, the method according to the present invention could also be applied for the analysis of biomolecules, biomarkers and/or bio-organisms (in addition to or instead of pathogens).

The verb ‘comprise’ and its conjugations as used in this patent document are understood to mean not only ‘comprise’, but to also include the expressions ‘contain’, ‘substantially contain’, ‘formed by’ and conjugations thereof.

1. A method for preparing a SERS substrate for analysis of at least one analyte in a fluid sample, comprising the steps of:
providing at least one fluid sample;
filtering the fluid sample through at least one catch filter, wherein the catch filter is configured to capture at least a fraction of at least one analyte if present in the fluid sample; and
filtering at least one solution comprising nanoparticles through the catch filter such that at least part of the catch filter will be coated with nanoparticles thereby forming a SERS substrate.

2. The method according to claim 1, wherein at least two steps are subsequent steps, and/or wherein at least two filtering steps are performed simultaneously.

3. (canceled)

4. The method according to claim 1, wherein at least part of the nanoparticles are metal nanoparticles, and/or wherein at least part of the nanoparticles is selected from the group of silver nickel, aluminium gold, platinum, palladium, titanium, copper, cobalt, zinc, and/or combinations thereof

5-9. (canceled)

10. The method according to claim 1, comprising the step of flushing at least the catch filter with a washing solution prior to at least one solution comprising nanoparticles is filtered through said catch filter.

11.-16 (canceled)

17. The method according to claim 1, wherein at least one coarse filter and/or at least one catch filter is comprised in a syringe filter.

18. The method according to claim 1, comprising the step of at least partially drying the catch filter after said catch filter is flushed with the solution comprising nanoparticles.

19. The method according to claim 1, comprising the step of using the nanoparticle coated catch filter as SERS substrate in a Raman spectroscopy analysis.

20. The method for pathogen analysis in a body fluid sample, comprising the steps of preparing a SERS substrate for pathogen analysis in a body fluid sample according to claim 1 and comprising the step of applying the nanoparticle coated catch filter as SERS substrate in a Raman spectroscopy analysis in order to analyse and/or identify any pathogens present.

21. (canceled)

22. A device for preparing a SERS substrate for analysis of at least one analyte in a fluid sample, comprising:
at least one filter assembly, wherein the filter assembly comprises:
at least one coarse filter configured to remove at least a fraction of particles from the body fluid sample; and
at least one catch filter configured to capture at least a fraction of at least one analyte from the fluid sample.

23. The device according to claim 22, wherein at least one coarse filter and at least one catch filter are mutually connected and/or connectable in a releasable manner.

24. The device according to claim 22, wherein at least one coarse filter comprises glass, a glass fiber mesh, glass wool, nylon, polyamide, cellulose acetate, polysulphone, teflon, cellulose and/or at least one hydrophilic polymer.

25. The device according to claim 22, wherein at least one coarse filter is configured to capture particles with a particle size larger than 2 μm.

26. The device according to claim 22, wherein at least one catch filter comprises glass, a glass fiber mesh, glass wool, nylon, polyamide, cellulose acetate, polysulphone, teflon, cellulose and/or at least one hydrophilic polymer, and/or wherein at least one catch filter is configured to filter particles with a particle size smaller than 5 μm.

27. (canceled)

28. The device according to claim 22, wherein at least one coarse filter and/or at least one catch filter is comprised in a syringe filter and/or wherein at least one coarse filter is received within a housing and/or wherein at least one catch filter is received within a housing.

29. (canceled)

30. The device according to claim 22, comprising at least one gripping structure for manual gripping of the device.

31. (canceled)

32. The device according to claim 22, comprising at least one fluid distribution structure for dividing fluid over the filter.

33. The device according to claim 22, comprising at least one guiding surface.

34. The method according to claim 1, comprising the step of filtering the fluid sample through at least one coarse filter, wherein the coarse filter is configured to remove at least a fraction of particles from the fluid sample.

35. The method according to claim 34, wherein at least one fluid sample is a body fluid sample and wherein at least one coarse filter is configured to filter at least part of white blood cells, red blood cells, epithelial cells and/or crystals from the body fluid sample.

36. The method according to claim 34, comprising the step of flushing the fluid sample through at least two coarse filters, wherein a first coarse filter is configured to remove at least a fraction of particles with a first particle size range and wherein a second coarse filter is configured to remove at least a fraction of particles with a second particle size range.
